(19) United States
(12) Patent Application Publication
(10) Pub. No.: US 2007/0061003 A1
(43) Pub. Date: Mar. 15, 2007

(54) Segmented ostial protection device

(75) Inventors: Ascher Shmulewitz, Tel Aviv (IL); Eyal Teichman, Hod-Hasharon (IL)

Correspondence Address: RISSMAN JOBSE HENDRICKS & OLIVERIO, LLP, ONE STATE STREET, SUITE 800, BOSTON, MA, 02109, US

(73) Assignee: Cappella, Inc., Westford, MA (US)

(21) Appl. No.: 11/252,224

(22) Filed: Oct. 17, 2005

Related U.S. Application Data
(63) Continuation-in-part of application No. 11/228,029, filed on Sep. 15, 2005, now abandoned.

(51) Int. Cl. A61F 2/06

(52) U.S. Cl. 623/1.16

(57) ABSTRACT

An intraluminal device for placement in a side-branch vessel associated with a main vessel via an ostial region. The intraluminal device includes an anchor portion, a cap portion and an articulating module. The anchor portion of the device is placed in the side branch vessel and the cap portion includes multiple protruding elements which are configured to extend into the ostial region. The articulating module includes a body and connectors, configured to provide large angular shifts over short distances and to absorb a portion of force causing a linear movement of the cap portion toward the anchor portion.

### RELATED APPLICATIONS

This application is a continuation-in-part of U.S. application Ser. No. 11/228,029 titled “Segmented Ostial Protection Device,” filed Sep. 15, 2005 the entire contents of which is incorporated herein.

### FIELD OF THE INVENTION

The invention relates to intraluminal devices for treatment at ostial regions of a vessel.

### BACKGROUND OF THE INVENTION

In today's society, many people suffer from a buildup of a plaque layer covering one or more segments of a coronary vessel where the lesion obstructs the flow of blood through the vessel. This buildup is referred to as a coronary lesion. Often, this condition is treated by placing medical devices or appliances within a patient for supporting the blood vessels or other lumens within the body that have been re-enlarged following cardio balloon angioplasty.

With regard to angioplasty, typically an endovascular or intraluminal implant known as a stent is placed within the blood vessel. A stent is usually tubular in shape and may have a lattice or connected-wire tubular construction. The stent is usually placed within the vessel in a compressed state and then allowed to expand.

The support structure of the stent is designed to prevent early collapse of a vessel that has been weakened and damaged by angioplasty. The support provided by the stent prevents the vessel from either closing, referred to as restenosis, or suffering spasms shortly after the angioplasty procedure, and has been shown to facilitate the healing of the damaged vessel wall, a process that occurs over a number of months. Self-expanding and balloon-expandable stents are well known.

During the healing process, inflammation caused by angioplasty and stent implant injury often causes smooth muscle cell proliferation and regrowth inside the stent, thus partially closing the flow channel, i.e., restenosis, thereby reducing or eliminating the beneficial effect of the angioplasty/stenting procedure. Blood clots may also form inside of the newly implanted stent due to the thrombotic nature of the stent surfaces, even when biocompatible materials are used to form the stent.

While large blood clots may not form during the angioplasty procedure itself, or immediately after the procedure, due to the current practice of injecting powerful anti-platelet drugs into the blood circulation, some thrombosis is always present, at least on a microscopic level on stent surfaces. This microscopic thrombosis is thought to play a significant role in the early stages of restenosis by establishing a biocompatible matrix on the surfaces of the stent whereupon smooth muscle cells may subsequently attach and multiply.

Stent coatings are known which contain bioactive agents that are designed to reduce or eliminate thrombosis or restenosis. Such bioactive agents may be dispersed or dissolved in either a bio-durable or bio-erodible polymer matrix that is attached to the surface of the stent wires prior to implant. After implantation, the bioactive agent diffuses out of the polymer matrix and into the surrounding tissue over a period lasting at least four weeks, and in some cases up to one year or longer, ideally matching the time course of restenosis, smooth muscle cell proliferation, thrombosis or a combination thereof.

Some coronary lesions may develop in coronary bifurcations, i.e., a bifurcated vessel including a main vessel associated via an ostial region with a side-branch vessel. Bifurcation lesions may be categorized according to the location of the lesion in the bifurcated vessel. In one example, a type 4a bifurcation lesion may refer to a lesion on the wall of the main vessel in proximity to the ostial region.

Treating bifurcation lesions, e.g., type 4a lesions, using the conventional methods described above, may result in at least part of the plaque layer “drifting” into the side-branch. This effect, commonly referred to as “the snow-plow effect,” may lead to a partial blockage of the side-branch, which may be treated by deploying one or more additional stents into the bifurcated vessel.

Conventional methods for treating bifurcation lesions may include deploying a first stent part in the main branch covering the side branch, and then inflating a “kissing balloon” and deploying a second stent part in the side branch, thereby to form a “T-stent” structure. Such methods as these, however, may result in the T-stent disrupting/obstructing the blood flow from the main vessel to the side branch.

Other stenting methods and/or specially designed bifurcation stents, for example, the Jostent® B stent, the Invatec Bifurcation stent, or the AST stent, may be relatively bulky and may have limited tractability, limited maneuverability and limited access to small caliber vessels. Moreover, other stenting methods do not provide adequate coverage at varying angles of bifurcation.

### SUMMARY OF THE INVENTION

In one embodiment, a device for implantation in a vessel comprises: an anchor portion having a proximal end, a distal end, and an anchor body connecting said proximal and distal ends, said anchor body comprising a series of struts configured to exert a radial force; a cap portion positioned proximal to said anchor portion; a plurality of protruding elements disposed at a proximal end of the cap portion; and an articulating module positioned proximal to the anchor portion and distal to the cap portion, the articulating module having a module body; at least one compressible cap connector connecting said module body to said cap portion; and at least one compressible anchor connector connecting said module body to said proximal end of the anchor portion.

In another embodiment, a device for implantation in a vessel comprises: an anchor portion having a proximal end, a distal end, and an anchor body connecting said proximal and distal ends, said anchor body comprising a series of struts configured to exert a radial force; a cap portion positioned proximal to said anchor portion; and articulating means, disposed between the anchor portion and the cap portion, for absorbing a portion of linear movement of the cap portion toward the anchor portion such that the absorbed portion of the linear movement of the cap portion is not transmitted to the anchor portion.

In another embodiment, a method of implanting a device in a vessel is provided. The device comprises: an anchor portion having a proximal end, a distal end, and an anchor body connecting said proximal and distal ends, said anchor body comprising a series of struts configured to exert a radial force; a cap portion positioned proximal to said anchor portion; a plurality of protruding elements disposed at a proximal end of the cap portion; and an articulating module positioned proximal to the anchor portion and distal to the cap portion, the articulating module having a module body; at least one compressible cap connector connecting said module body to said cap portion; and, and at least one compressible anchor connector connecting said module body to said proximal end of the anchor portion. The method comprises: positioning the anchor portion of the device in a side branch vessel such that the struts of the anchor body exert a radial force on the side branch vessel and the cap portion is positioned at a bifurcation location between the side branch vessel and a main branch vessel; exerting a force upon the cap portion to urge the cap portion a linear distance toward the anchor portion; and absorbing a portion of the linear distance with the articulating module such that the anchor portion substantially remains in position within the side branch vessel.

### BRIEF DESCRIPTION OF THE DRAWINGS

The above and further advantages of the invention may be better understood by referring to the following description in conjunction with the accompanying drawings in which:

FIG. 1 is a schematic illustration of a bifurcated vessel including a main vessel and a side branch vessel;

FIG. 2A is a schematic illustration of an intraluminal device in accordance with embodiments of the present invention;

FIG. 2B is a schematic illustration of an articulating module from the intraluminal device of FIG. 2A;

FIG. 3 is a perspective view illustration of an intraluminal device in accordance with exemplary embodiments of the invention;

FIG. 4 is an illustration of an intraluminal device in a flattened view, showing the geometric configuration and patterns in accordance with exemplary embodiments of the present invention;

FIG. 5 is an illustration of an intraluminal device in a flattened view, showing the geometric configuration and patterns in accordance with other embodiments of the present invention;

FIG. 6 is an illustration of an intraluminal device in a flattened view, showing the geometric configuration and patterns in accordance with yet other embodiments of the present invention;

FIG. 7A is a perspective view illustration of an intraluminal device in accordance with one embodiment of the present invention;

FIG. 7B is an illustration of the intraluminal device of FIG. 7A in a flattened view, showing the geometric configuration and patterns in accordance with embodiments of the present invention;

FIGS. 8A-8H are schematic illustrations showing the steps of a method of deploying an intraluminal device in accordance with exemplary embodiments of the present invention;

FIGS. 9A-9D are schematic illustrations of the steps of a method of increasing side branch vessel access following the steps of deployment of FIGS. 8A-8H;

FIGS. 10A-10D are schematic illustrations showing steps of a method for situating a device in accordance with one embodiment of the present invention; and

FIGS. 11A and 11B are drawings of a body portion in a relaxed and bent configuration, respectively.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of the invention. It will be understood by those of ordinary skill in the art that the present invention may be practiced without these specific details. In other instances, well-known methods, procedures, components and structures may not have been described in detail so as not to obscure the present invention.

Embodiments of the invention may include an intraluminal device configured to selectively protect at least part of a predetermined region, e.g., an ostial region, of a bifurcated vessel and/or to dispense medication substantially uniformly across at least part of the predetermined region, as described below.

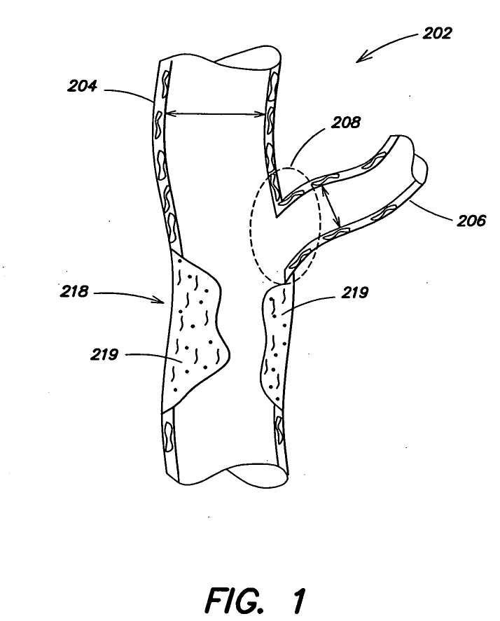

Reference is now made to FIG. 1, which schematically illustrates a bifurcated vessel 202 including a main vessel 204 and a side branch vessel 206 extending from main vessel 204. Bifurcated vessel 202 may include a target tissue, for example, a diseased segment (a “lesion”), which may include a plaque layer 219 obstructing the flow of blood through the diseased segment of the vessel. The lesion may be located along at least part of main vessel 204, side branch vessel 206 and/or an ostial region 208 between side-branch vessel 206 and main vessel 204. For example, a type 4a bifurcation lesion 218 may be located in main vessel 204 in proximity to ostial region 208.

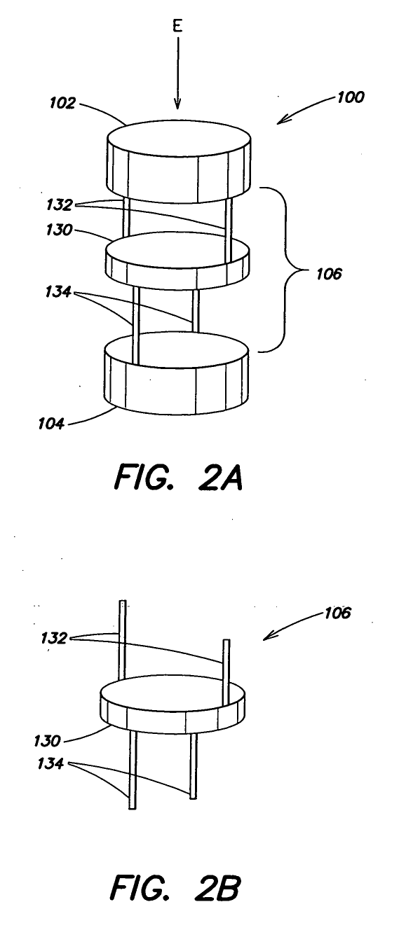

Reference is now made to FIG. 2A, which illustrates a schematic view of an intraluminal device 100 and to FIG. 2B, which illustrates a schematic view of an articulating module 106 of intraluminal device 100. Intraluminal device 100 includes a cap portion 102, an anchor portion 104, and an articulating module 106. Anchor portion 104 is configured to fit into side-branch vessel 206. Cap portion 102 is configured to selectively protect at least part of ostial region 208. Articulating module 106 flexibly connects anchor portion 104 to cap portion 102, such that various angles of articulation are possible between each of the three portions, as described in detail below.

Specifically, intraluminal device 100 is able to provide large angular shifts over short axial distances, due to its design characteristics.

Articulating module 106 includes cap connectors 132 connecting a body 130 of articulating module 106 to cap portion 102, and anchor connectors 134 connecting body 130 to anchor portion 104. In some embodiments, cap connectors 132 include two connectors, separated from each other by 180 degrees around body 130, and anchor connectors 134 include two connectors, separated from each other by 180 degrees around body 130, and further positioned at approximately 90 degrees from cap connectors 132 around body 130. Thus, cap connectors 132 may be flexed back and forth in one direction or plane and anchor connectors 134 may be flexed back and forth in another direction or plane which is orthogonal to the direction of flexing of cap connectors 132, providing multiple directional flexibility overall by articulating module 106. In some embodiments, flexing of cap connectors 132 and anchor connectors 134 is variable, such that either one or both of cap connectors 132 and anchor connectors 134 can be flexed in multiple directions.

In some embodiments, cap connectors 132 and anchor connectors 134 are pre-shaped for specific angles, requiring less force for flexing at the specific angles.

In some embodiments, only one cap connector 132 and/or one anchor connector 134 is used. Body 130 can be of various designs and geometries, but should be designed such that it can be crimped to a smaller diameter and expanded upon deployment of intraluminal device 100. Examples of such designs are described more fully hereinbelow.

A further function of articulating module 106 is to provide a spring-like mechanism for correction of axial, i.e., longitudinal, positioning of cap portion 102 within a vessel. Thus, when a force is exerted on the cap portion 102, causing the cap portion 102 to move in a direction E along the longitudinal axis of the device 100, i.e., toward the anchor portion 304 as shown in FIG. 2A, a portion of the linear motion or movement is absorbed by the articulating module 106 and not passed along to the anchor portion 104. This “energy absorbing” or linear position adjustment function facilitates placement of the device 100, as will be described in more detail below. The force or linear movement is absorbed by operation of the body 130, cap connectors 132 and the anchor connectors 134 either individually or in combination with one another.

Intraluminal device 100 may be configured to protect the ostial region 208 and/or the side branch vessel 206 by selectively covering at least part of an inner wall of the ostial region 208 in order, for example, to prevent the plaque layer 219 or parts thereof from migrating into the side branch vessel 206 by the snow-plow effect, which may result from applying the angioplasty device, as described below.

According to exemplary embodiments of the invention, intraluminal device 100 may be formed of a generally elastic, super-elastic, in-vivo stable and/or “shape-memorizing” material, i.e., a material able to be initially formed in a desired shape, e.g., during an initial procedure performed at relatively high temperature, to be deformed, e.g., compressed, and to assume the desired shape in which it was previously shaped.

Intraluminal device 100 may be formed of Nickel-Titanium alloy (“nitinol”) wire which possesses both super-elastic and shape-memorizing properties. The wire may have a diameter of between 30 and 300 micrometers. In other embodiments, biocompatible non-elastic materials, such as stainless steel, for example, may be used.

In some embodiments, intraluminal device 100 is formed from a wire. In other embodiments, intraluminal device 100 is cut from a single tube. Intraluminal device 100 may be formed from a single piece of material or may be assembled in sections. In an alternative embodiment, cap portion 102 may be of a different material than anchor portion 104. Cap portion 102 may be formed from any compliant material known to one of ordinary skill in the art, e.g., a polymeric material. Further, cap portion 102 may be formed from a non-compliant material.

According to exemplary embodiments of the invention, at least part of intraluminal device 100 may be coated with a layer of a desired medication or a material having desired properties to carry and subsequently apply and/or dispense a desired medication. Anchor portion 104 and/or cap portion 102 may be coated with a controlled-release polymer and/or drug, as known in the art, for reducing the probability of undesired side effects, e.g., restenosis. The restenosis may occur as a result of a percutaneous procedure performed on the bifurcated vessel 202, e.g., including insertion of an angioplasty device into the bifurcated vessel 202.

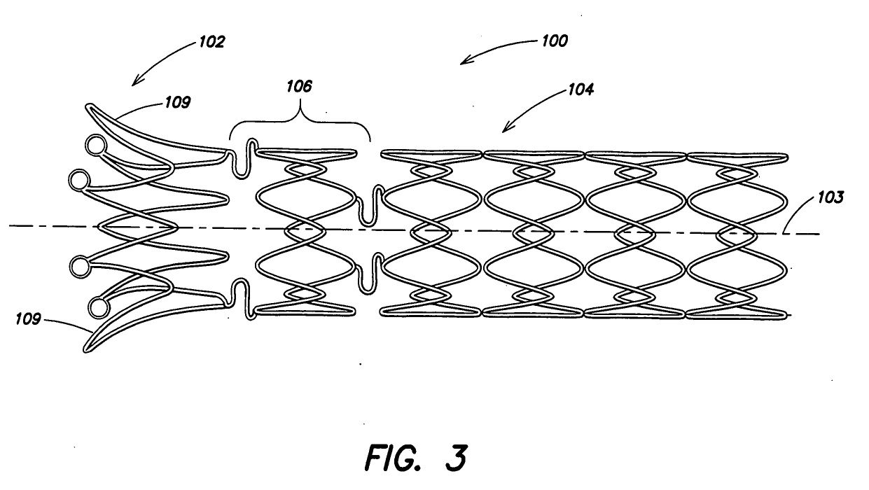

Reference is now made to FIG. 3, which is a perspective illustration of intraluminal device 100, in accordance with exemplary embodiments of the invention. Intraluminal device 100 includes anchor portion 104, cap portion 102, and articulating module 106 connecting anchor portion 104 to cap portion 102.

According to exemplary embodiments of the invention, anchor portion 104 may have a generally tubular, e.g., spring-like, structure, which may be circularly symmetric with respect to a central axis 103. In other embodiments, anchor portion 104 has a geometric configuration of struts, as described in detail below. In some embodiments, anchor portion 104 has a generally conical structure, wherein a distal portion thereof has a smaller diameter than a proximal portion thereof. Anchor portion is 104 is configured to hold intraluminal device 100 in place in the vessel, preventing shifting of the device. An outer diameter of anchor portion 104 may be compatible with, i.e., approximately equal to or slightly larger than, an inner diameter of the side branch vessel 206. According to some exemplary embodiments of the invention, the outer diameter of anchor portion 104 may be substantially constant along the central axis 103. According to other embodiments, the outer diameter of anchor portion 104 may vary along the central axis 103, e.g., in order to enable an improved positioning and/or “anchoring” of the anchor portion 104 with respect to the side branch 206 and/or to ease the insertion of the intraluminal device 100 into the side branch. For example, anchor portion 104 may have a generally conical shape, i.e., the outer diameter of anchor portion 104 may monotonically increase or decrease along central axis 103.

According to exemplary embodiments of the invention, cap portion 102 includes multiple protruding elements 109 extending in a proximal direction. In exemplary embodiments, multiple protruding elements 109 are configured to extend into or in a direction of ostial region 208. The number of multiple protruding elements 109 is chosen based on the particular anatomy in which intraluminal device 100 is to be placed. Upon deployment of intraluminal device 100, multiple protruding elements 109 extend outwardly, forming a trumpet shape, and protecting areas of ostial region 208 which are frequently not adequately protected due to the configurations of known intraluminal devices.

Articulating module 106 provides additional flexibility to intraluminal device 100, by providing a configuration to allow for angular shifts, as well as axial positioning, as described below in further detail.

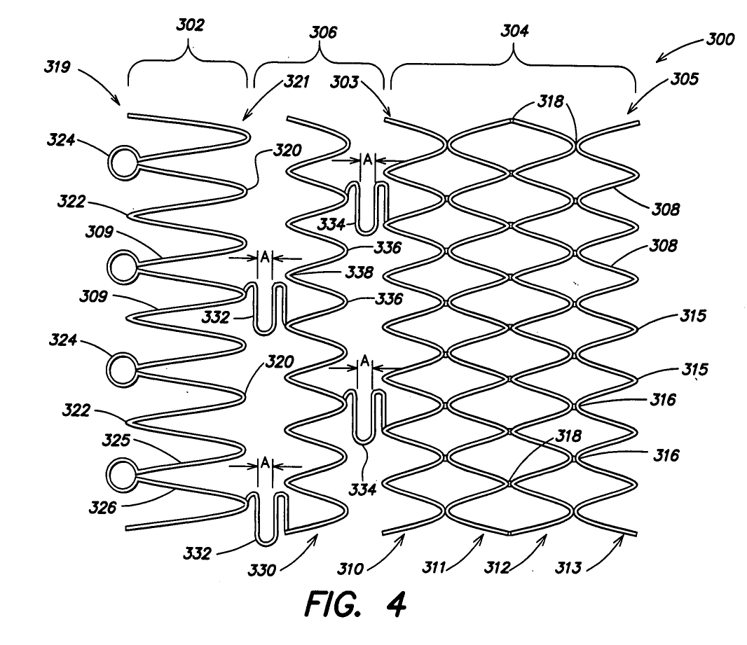

45 Reference is now made to FIG. 4, which is an illustration of an intraluminal device 300 in a flattened view, showing the geometric configuration and patterns in accordance with exemplary embodiments of the present invention. An anchor portion 304 has an anchor portion proximal end 303 and an anchor portion distal end 305, wherein anchor portion proximal end 303 is at least partially connected to other portions of intraluminal device 300 as described hereinbelow. Anchor portion 304 is comprised of struts or supporting elements 308, which are interconnected to provide support to an inner portion of the side branch vessel 206. In some embodiments, supporting elements 308 form a uniform or repeating cell pattern, such as repeating diamond shapes, hexagonal shapes, or any other pattern. In alternative embodiments, supporting elements 308 form non-uniform patterns, having variations in pattern dimensions and/or strut characteristics. In one embodiment, supporting elements 308 are configured in a series of interconnected columns, for example, columns 310-313 shown in FIG. 4.

It should be readily apparent that the number of columns 310-313 may vary, and that the number of columns shown and described herein with respect to the present embodiment is for illustrative purposes only. Each column 310-313 has a sinusoidal pattern having peaks 315 and valleys 316, wherein peaks 315 are defined as elements protruding in a direction facing anchor portion distal end 305 and valleys 316 are defined as elements protruding in a direction facing anchor portion proximal end 303. Adjacent columns are 180 degrees out of phase in their sinusoidal patterns, such that a peak 315 of one column, for example column 310, is in line with a valley 316 of an adjacent column, for example column 311. This configuration can be repeatedly applied to additional columns, such that any desired number of columns may be included. Columns 310-313 are connected to one another at contact areas 318 between peaks 315 of one column and valleys 316 of an adjacent column. In alternative embodiments, adjacent columns are in phase with one another, or out of phase by other degrees. A length of anchor portion 304 may be in a range of 4-20 mm when in an unexpanded state, and a diameter in a range of 2-6 mm in a fully expanded state.

A cap portion 302 includes multiple protruding elements 309 configured, for example, in a sinusoidal pattern having cap peaks 320 and cap valleys 322, wherein cap peaks 320 are defined as elements facing a distal side 321 of cap portion 302 and cap valleys 322 are defined as elements facing a proximal side 319 of cap portion 302. Cap peaks 320 and cap valleys 322 are connected by upper segments 325 and lower segments 326 which are repeatedly angled in one direction and in the opposite direction, such that upper segments 325 are connected to lower segments 326 alternatingly at proximal side 319 forming cap valleys 322 and at a distal side 321 forming cap peaks 320. In alternative embodiments, protruding elements 309 are comprised of other patterns, including non-angled upper and lower segments, rounded, squared or any other suitable configuration. In exemplary embodiments, multiple protruding elements 309 are longer than supporting elements 308 of individual columns of anchor portion 304, and are configured to extend into or in a direction of ostial region 208. Some of protruding elements 309 further include tip portions 324 at their proximal ends. In one embodiment, only some of protruding elements 309 (such as every alternate one, for example) include a tip portion 324. In other embodiments, every protruding element 309 includes a tip portion 324. Tip portions 324 provide additional surface area for delivery of medication, and are also suitable for placing of markers thereon. In some embodiments, multiple protruding elements 309 are in a range of 1-6 mm in length. After shaping, a diameter defined by cap peaks 320 may be in range of 3-10 mm.

An articulating module 306 is provided between anchor portion 304 and cap portion 302, and includes a body 330, cap connectors 332 and anchor connectors 334. A purpose of articulating module 306 is to provide a small radius of curvature between anchor portion 304 and cap portion 302, so that intraluminal device 300 can bend at many different angles without significant additional rotation. A further purpose of articulating module 306 is to provide a spring-like mechanism for correction of axial positioning of cap portion 302 within a vessel. Thus, a portion of a force exerted on, for example, the cap portion 302 that causes the cap portion 302 to move in a direction along the longitudinal axis of the device 100 toward the anchor portion 304, is absorbed by the articulating module 306 and the linear motion or movement is not passed along to the anchor portion 304. This “energy absorbing” or linear position compensation operation facilitates placement of the device 300, as will be described in more detail below. The energy is absorbed by operation of the body 330, cap connectors 332 and the anchor connectors 334 either individually or in combination with one another.

Body 330 may have a similar geometric pattern or configuration as anchor portion 304, or may have a different pattern or configuration. A length of body 330 is minimized so as to ensure maximum flexing capabilities. For example, a length of body 330 may be in a range of 0.5-4 mm. In one embodiment, body 330 includes a row of interconnecting struts having a sinusoidal pattern having peaks 336 and valleys 338, wherein peaks 336 are defined as elements protruding in a direction facing anchor portion 304 and valleys 338 are defined as elements protruding in a direction facing cap portion 302, as shown in FIG. 4.

In the embodiment shown in FIG. 4, anchor connectors 334 are disposed between peaks 336 of articulating module 306 and valleys 316 of anchor portion 304. Furthermore, cap connectors 332 are disposed between valleys 338 of articulating module 306 and peaks 320 of cap portion 302. In exemplary embodiments, anchor connectors 334 are spaced apart from one another so as to provide a high degree of flexibility between articulating module 306 and anchor portion 304, and cap connectors 332 are spaced apart from one another so as to provide a high degree of flexibility between articulating module 306 and cap portion 302. For example, anchor connectors 334 may be placed on one of every five or six peaks 336 of articulating module 306, and cap connectors 336 may be placed on one of every five or six valleys 338 of articulating module 306, such that anchor connectors 334 and cap connectors 336 are alternatingly positioned along body 330. In some embodiments, the struts of body 330 of articulating module 306 are shorter than the struts of protruding elements 309 of cap portion 302. In some embodiments, anchor connectors 334 and cap connectors 336 are straight connectors. In other embodiments, anchor connectors 334 and cap connectors 336 are curved connectors, spiral connectors, or S-shaped connectors, as shown in FIG. 4. In some embodiments, anchor connectors 334 and cap connectors 336 are pre-shaped. In some embodiments, anchor connectors 334 do not have the same configuration as cap connectors 336.

The linear compensation function of the device 300 operates by function of the cap connectors 332, the anchor connectors 334 and the body 330. As shown in FIG. 4, each of the cap connectors 332 and the anchor connectors 334 includes a connector space A. When a force is exerted on the cap portion 302, the space A closes, i.e., is compressed, and the portions of the cap connectors 332 or anchor connectors 334 are urged toward one another to accommodate movement of the cap portion 302. A component of the total linear absorption or compensation provided by the cap connectors 332 and the anchor connectors 334 is, therefore, 2*A.

Further, linear compensation is provided by the body 330. When the force on, or linear movement of, the cap portion 302 is conveyed to the body 330 via the cap connectors 332, the body 330 will bend in a manner analogous to straight beam bending.

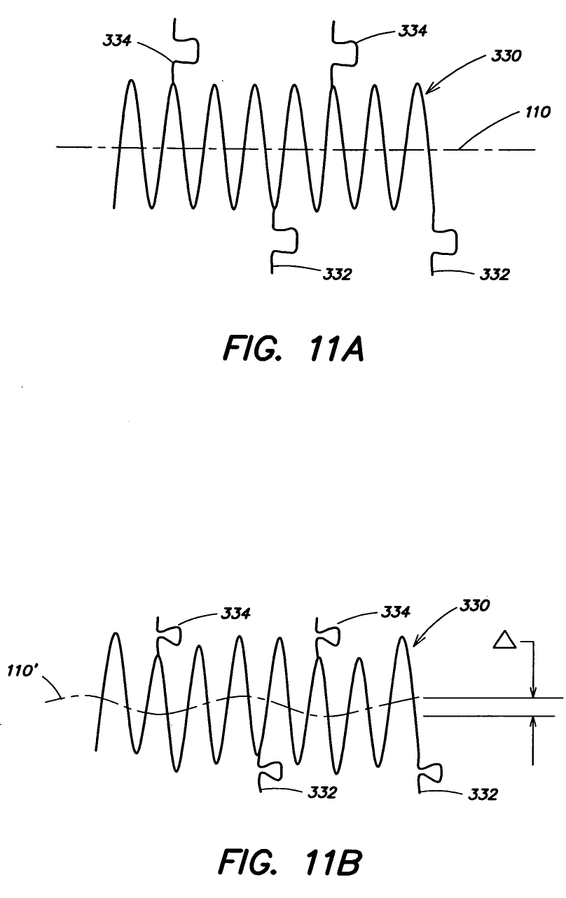

When no force is applied, the body 330 has a straight neutral circumferential axis or line1 10 as shown in FIG. 11A. Upon the exertion of a force on to the body 330, each of the cap connectors 332 and the anchor connectors 334 compresses and the body 330 acquires a curved neutral circumferential axis or line 110′, as shown in FIG. 11B. A peak-to-peak value A of the curved neutral circumferential axis or line 110′, as shown in FIG. 11B, is the portion of the linear displacement absorbed by the body 330. It should be noted that the drawings in FIGS. 11A and 11B are for explanatory purposes only and not meant to limit the present invention. The representations are not to scale and the amount of bend has been exaggerated for ease of explanation.

A total linear absorption value L can then be expressed as L=2*A+Δ. In an exemplary embodiment L is in the range of 1-2 mm.

Of course, one of ordinary skill in the art will understand that there is an upper limit to how much linear movement or displacement of the cap portion 302 can be absorbed by the articulating module 306.

It should be readily apparent that different numbers of connectors as well as different configurations of struts, connectors, and protruding elements and patterns related thereto may vary, and that all such possibilities are within the scope of the present invention. It should also be apparent that the view shown herein represents a structure of intraluminal device 300 prior to shaping. Intraluminal device 300 may subsequently be shaped in accordance with known techniques, such that multiple protruding elements 309 are outwardly projected, forming a substantially trumpet-like configuration. A trumpet shape formed by shaping of intraluminal device 300 may have a radius of curvature in a range of 0.5-10 mm, and an angle of bending in a range of 90-180 degrees. Further, the shape and spacing of the body 330, cap connectors 332 and the anchor connectors 334 can be adjusted to modify the total linear absorption value L.

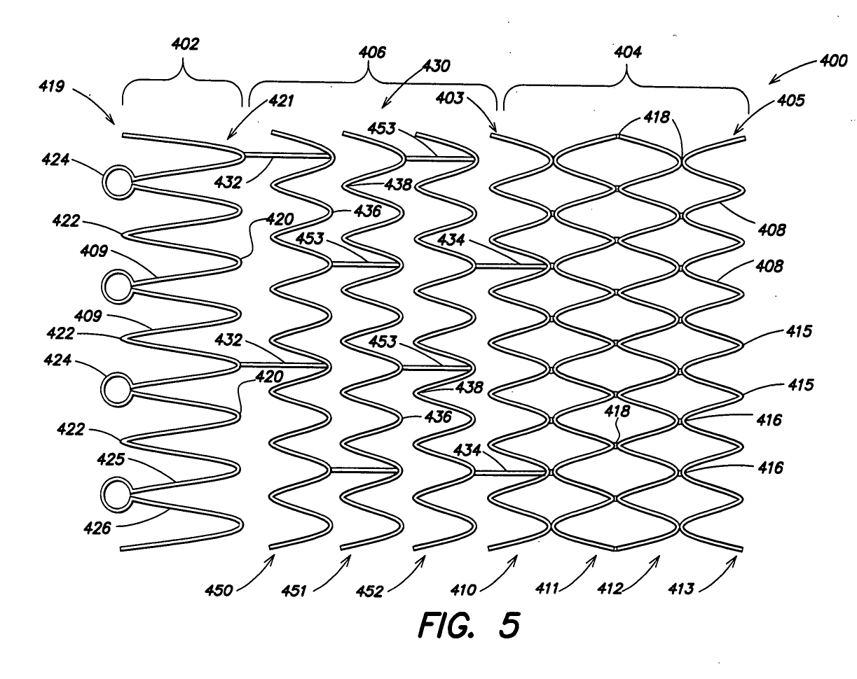

Reference is now made to FIG. 5, which is an illustration of an intraluminal device 400 in a flattened view, showing the geometric configuration and patterns in accordance with exemplary embodiments of the present invention. An anchor portion 404 has an anchor portion proximal end 403 and an anchor portion distal end 405, wherein anchor portion proximal end 403 is connected to other portions of intraluminal device 400 as described hereinbelow. Anchor portion 404 is comprised of struts or supporting elements 408, which are interconnected to provide support to an inner portion of the side branch vessel 206. In some embodiments, supporting elements 408 form a uniform or repeating cell pattern, such as repeating diamond shapes, hexagonal shapes, or any other pattern. In alternative embodiments, supporting elements 408 form non-uniform patterns, having variations in pattern dimensions and/or strut characteristics. In one embodiment, supporting elements 408 are configured in a series of interconnected columns, for example columns 410-413 shown in FIG. 5.

It should be readily apparent that the number of columns 410-413 may vary, and that the number of columns shown and described herein with respect to the present embodiment is for illustrative purposes only. Each column 410-413 has a sinusoidal pattern having peaks 415 and valleys 416, wherein peaks 415 are defined as elements protruding in a direction facing anchor portion distal end 405 and valleys 416 are defined as elements protruding in a direction facing anchor portion proximal end 403. Adjacent columns are 180 degrees out of phase in their sinusoidal patterns, such that a peak 415 of one column, for example column 410, is adjacent to a valley 416 of an adjacent column, for example column 411. This configuration can be repeatedly applied to additional columns, such that any desired number of columns may be included. Columns 410-413 are connected to one another at contact areas 418 between peaks 415 of one column and valleys 416 of an adjacent column. In alternative embodiments, adjacent columns are in phase with one another, or out of phase by other degrees. A length of anchor portion 404 may be in a range of 4-20 mm when in an unexpanded state, and a diameter in a range of 2-6 mm in a fully expanded state.

A cap portion 402 includes multiple protruding elements 409 configured, for example, in a sinusoidal pattern having cap peaks 420 and cap valleys 422, wherein cap peaks 420 are defined as elements facing a distal side 421 of cap portion 402 and cap valleys 422 are defined as elements facing a proximal side 419 of cap portion 402. Cap peaks 420 and cap valleys 422 are connected by upper segments 425 and lower segments 426 which are repeatedly angled in one direction and in the opposite direction, such that upper segments 425 are connected to lower segments 426 alternatingly at proximal side 419 forming cap valleys 422 and at a distal side 421 forming cap peaks 420. In alternative embodiments, protruding elements 409 are comprised of other patterns, including non-angled upper and lower segments, rounded, squared or any other suitable configuration. In exemplary embodiments, multiple protruding elements 409 are longer than supporting elements 408 of individual columns of anchor portion 404, and are configured to extend into or in a direction of ostial region 208. Some of protruding elements 409 further include tip portions 424 at their proximal ends. In one embodiment, only some of protruding elements 409 (such as every alternate one, for example) include a tip portion 424. In other embodiments, every protruding element 409 includes a tip portion 424. Tip portions 424 provide additional surface area for delivery of medication, and are also suitable for placing of markers thereon. In some embodiments, multiple protruding elements 409 are in a range of 1-6 mm in length. After shaping, a diameter defined by cap peaks 420 may be in a range of 3-10 mm.

An articulating module 406 is provided between anchor portion 404 and cap portion 402, and includes a body 430, cap connectors 432 and anchor connectors 434. A purpose of articulating module 406 is to provide a small radius of curvature between anchor portion 404 and cap portion 402, so that intraluminal device 400 can bend at many different angles without significant additional rotation. A further purpose of articulating module 406 is to provide a spring-like mechanism for correction of axial positioning of cap portion 402 within a vessel. Thus, a portion of a force exerted on, for example, the cap portion 402 that causes the cap portion 402 to move in a direction along the longitudinal axis of the device 400 toward the anchor portion 404, is absorbed by the articulating module 406 and the linear motion or movement is not passed along to the anchor portion 404. This “energy absorbing” or linear position compensation operation facilitates placement of the device 400, as will be described in more detail below. The energy is absorbed by operation of the body 430, cap connectors 432 and the anchor connectors 434 either individually or in combination with one another.

Body 430 may have a similar geometric pattern or configuration as anchor portion 404, or may have a different pattern or configuration. A length of body 430 is minimized so as to ensure maximum flexing capabilities. For example, a length of body 430 may be in a range of 0.5-4 mm. In one embodiment, body 430 includes multiple rows of interconnecting struts having a sinusoidal pattern having peaks 436 and valleys 438, wherein peaks 436 are defined as elements protruding in a direction facing anchor portion 404 and valleys 438 are defined as elements protruding in a direction facing cap portion 402, as shown in FIG. 5. In this embodiment, adjacent columns 450-452 are connected to one another by articulating body connectors 453. Articulating body connectors 453 are configured to connect peaks 436 of adjacent articulating body columns to one another. In this configuration, peaks 436 of each of columns 450-452 and valleys 438 of each of columns 450-452 are substantially aligned, such that overlapping of adjacent portions of articulating body 430 is minimized upon flexing. In an alternative embodiment, articulating body connectors 453 are configured to connect valleys 438 of adjacent articulating body columns to one another. In exemplary embodiments, articulating body connectors 453 are spaced apart from one another so as to provide a high degree of flexibility between articulating body columns 450-452.

For example, articulating body connectors 453 may be placed on one of every four or five peaks in an individual articulating body column for increased flexibility. Articulating body connectors 453 may be straight connectors, as shown in FIG. 5, or may be curved, spiral or S connectors or any other suitable connector.

In the embodiment shown in FIG. 5, anchor connectors 434 are disposed between peaks 436 of a distal column 452 of articulating module 406 and valleys 416 of anchor portion 404. Furthermore, cap connectors 436 are disposed between valleys 438 of a proximal column 450 of articulating module 406 and peaks 420 of cap portion 402. In exemplary embodiments, anchor connectors 434 are spaced apart from one another so as to provide a high degree of flexibility between articulating module 406 and anchor portion 404, and cap connectors 432 are spaced apart from one another so as to provide a high degree of flexibility between articulating module 406 and cap portion 402. For example, anchor connectors 434 may be placed on one of every five or six peaks 436 of distal column 437 of articulating module 406, and cap connectors 436 may be placed on one of every five or six valleys 438 of proximal column 439 of articulating module 406, such that anchor connectors 434 and cap connectors 436 are alternatingly positioned along body 430. In some embodiments, the struts of body 430 of articulating module 406 are shorter than the struts of protruding elements 409 of cap portion 402. In some embodiments, anchor connectors 434 and cap connectors 436 are straight connectors, as shown in FIG. 5. In other embodiments, anchor connectors 434 and cap connectors 436 are curved connectors, spiral connectors, or S-shaped connectors. In some embodiments, anchor connectors 434 and cap connectors 436 are pre-shaped. In some embodiments, anchor connectors 434 do not have the same configuration as cap connectors 436.

The linear compensation function of the device 400 operates primarily by function of the body 430. As shown in FIG. 5, each of the cap connectors 432 and the anchor connectors 434 is a straight connector and, therefore, connector space A=0 and does not provide a linear compensation function. When the force, or linear movement, is conveyed to the body 430, the body 430 operates in a fashion that is analogous to that described with reference to FIGS. 11A and 11B.

The total linear absorption value L can then be expressed as L=2*A+Δ and, where A=0, L=Δ. In the exemplary embodiment of FIG. 5, L is in the range of 1-1.5 mm.

It should be readily apparent that different numbers of connectors as well as different configurations of struts, connectors, and protruding elements and patterns related thereto may vary, and that all such possibilities are within the scope of the present invention. It should also be apparent that the view shown herein represents a structure of intraluminal device 400 prior to shaping. Intraluminal device 400 may subsequently be shaped in accordance with known techniques, such that multiple protruding elements 409 are outwardly projected, forming a substantially trumpet-like configuration. A trumpet shape formed by shaping of intraluminal device 400 may have a radius of curvature in a range of 0.5-10 mm, and an angle of bending in a range of 90-180 degrees. Further, the shape and spacing of the body 430, cap connectors 432 and the anchor connectors 434 can be adjusted to modify the total linear absorption value L.

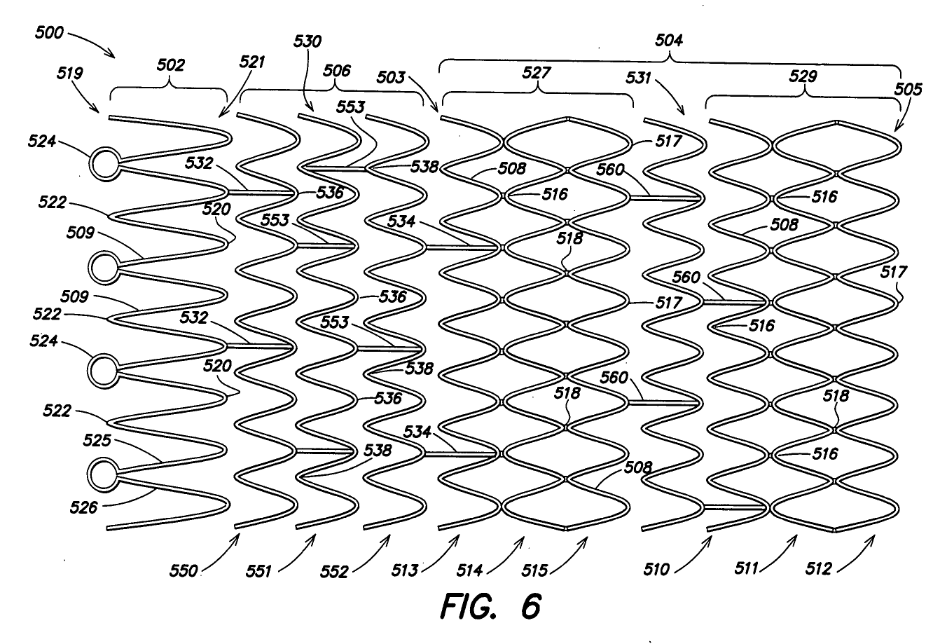

Reference is now made to FIG. 6, which is an illustration of an intraluminal device 500 in a flattened view, showing the geometric configuration and patterns in accordance with alternative embodiments of the present invention. An anchor portion 504 has an anchor portion proximal end 503 and an anchor portion distal end 505, wherein anchor portion proximal end 503 is connected to other portions of intraluminal device 500 as described hereinbelow. Anchor portion 504 includes a first anchor section 527, a second anchor section 529, and an anchor-connecting segment 531 connecting first and second anchor sections 527 and 529. First anchor section 527 and second anchor section 529 are comprised of struts or supporting elements 508, which are interconnected to provide support to an inner portion of the side branch vessel 206.

Anchor connecting segment 531 includes at least one row of struts which is flexibly connected to each of first and second anchor sections 527 and 529, thereby providing additional flexibility to anchor portion 504. This type of design may be useful in a branch vessel having a curved or tortuous geometry, for example. Additionally, it may be possible to use only the most distal portion (second anchor section 529 in FIG. 6) for initial anchoring, and the remainder of anchor portion 104 for subsequent anchoring during a procedure. Anchor connecting segment 531 is connected to first and second anchor sections 527 and 529 via anchor segment connectors 560. In exemplary embodiments, anchor segment connectors 560 are spaced apart from one another so as to provide a high degree of flexibility between first and second anchor sections 527 and 529. For example, anchor segment connectors 560 may be placed on one of every four or five peaks of distal column 515 of first anchor section 527 for increased flexibility.

Anchor segment connectors 560 may be straight connectors, as shown in FIG. 6, or may be curved, spiral or S connectors or any other suitable connector.

In some embodiments, supporting elements 508 form a uniform or repeating cell pattern, such as repeating diamond shapes, hexagonal shapes, or any other pattern. In alternative embodiments, supporting elements 508 form non-uniform patterns, having variations in pattern dimensions and/or strut characteristics. In one embodiment, supporting elements 508 of first and second anchor sections 527 and 529 are configured in two series of interconnected columns 510-512, 513-515.

It should be readily apparent that the number of columns 510-515 and sections may vary, and that the number of columns and sections shown and described herein with respect to the present embodiment is for illustrative purposes only. Each column 510-515 has, for example, a sinusoidal pattern having peaks 515 and valleys 516, wherein peaks 515 are defined as elements protruding in a direction facing anchor portion distal end 505 and valleys 516 are defined as elements protruding in a direction facing anchor portion proximal end 503. Adjacent columns are, for example, 180 degrees out of phase in their sinusoidal patterns, such that a peak 515 of one column, for example column 510, is adjacent to a valley 516 of an adjacent column, for example column 511. This configuration can be repeatedly applied to additional columns, such that any desired number of columns may be included. Columns 510-512 and 513-515 are connected to one another at contact areas 518 between peaks 515 of one column and valleys 516 of an adjacent column. In alternative embodiments, adjacent columns are in phase with one another, or out of phase by other degrees. A length of anchor portion 504 may be in a range of 4-20 mm when in an unexpanded state, and a diameter in a range of 2-6 mm in a fully expanded state.

A cap portion 502 includes multiple protruding elements 509 configured in a sinusoidal pattern having cap peaks 520 and cap valleys 522, wherein cap peaks 520 are defined as elements facing a distal side 521 of cap portion 502 and cap valleys 522 are defined as elements facing a proximal side 519 of cap portion 502. Cap peaks 520 and cap valleys 522 are connected by upper segments 525 and lower segments 526 which are repeatedly angled in one direction and in the opposite direction, such that upper segments 525 are connected to lower segments 526 alternatingly at proximal side 519 forming cap valleys 522 and at distal side 521 forming cap peaks 520. In alternative embodiments, protruding elements 509 are comprised of other patterns, including non-angled upper and lower segments, rounded, squared or any other suitable configuration. In exemplary embodiments, multiple protruding elements 509 are longer than supporting elements 508 of individual columns of anchor portion 504, and are configured to extend into ostial region 208. Some of protruding elements 509 further include tip portions 524 at their proximal ends. In one embodiment, only some of protruding elements 509 (such as every alternate one, for example) include a tip portion 524. In other embodiments, every protruding element 509 includes a tip portion 524.

Tip portions 524 provide additional surface area for delivery of medication, and are also suitable for placing of markers thereon. In some embodiments, multiple protruding elements 509 are in a range of 1-6 mm in length. After shaping, a diameter defined by cap peaks 520 may be in a range from 3-10 mm.

An articulating module 506 is provided between anchor portion 504 and cap portion 502, and includes a body 530, cap connectors 532 and anchor connectors 534. A purpose of articulating module 506 is to provide a small radius of curvature between anchor portion 504 and cap portion 502, so that intraluminal device 500 can bend at many different angles without significant additional rotation. A further purpose of articulating module 506 is to provide a spring-like mechanism for correction of axial positioning of cap portion 502 within a vessel. Thus, a portion of a force exerted on, for example, the cap portion 502 that causes the cap portion 502 to move in a direction along the longitudinal axis of the device 500 toward the anchor portion 504, is absorbed by the articulating module 506 and not passed along to the anchor portion 504. This “energy absorbing” or linear positioning compensation operation facilitates placement of the device 500, as will be described in more detail below. The energy is absorbed by operation of the body 530, cap connectors 532 and the anchor connectors 534 either separately or in combination/sub-combination with one another.

Body 530 may have a similar geometric pattern or configuration as anchor portion 504, or may have a different pattern or configuration. A length of body 530 is minimized so as to ensure maximum flexing capabilities. For example, a length of body 530 may be in a range of 0.5-4 mm. In one embodiment, body 530 includes multiple rows of interconnecting struts having a sinusoidal pattern having peaks 536 and valleys 538, wherein peaks 536 are defined as elements protruding in a direction facing anchor portion 504 and valleys 538 are defined as elements protruding in a direction facing cap portion 502, as shown in FIG. 6. In this embodiment, adjacent columns 550-552 are connected to one another by articulating body connectors 553. Articulating body connectors 553 are configured to connect peaks 536 of adjacent articulating body columns to one another. In an alternative embodiment, articulating body connectors 553 are configured to connect valleys 538 of adjacent articulating body columns to one another. In exemplary embodiments, articulating body connectors 553 are spaced apart from one another so as to provide a high degree of flexibility between articulating body columns 550-552. For example, articulating body connectors 553 may be placed on one of every four or five peaks in an individual articulating body column for increased flexibility. Articulating body connectors 553 may be straight connectors, as shown in FIG. 6, or may be curved, spiral or S connectors or any other suitable connector.

In the embodiment shown in FIG. 6, anchor connectors 534 are disposed between peaks 536 of a distal column 552 of articulating module 506 and valleys 516 of anchor portion 504. Furthermore, cap connectors 536 are disposed between valleys 538 of a proximal column 550 of articulating module 506 and peaks 522 of cap portion 502. In exemplary embodiments, anchor connectors 534 are spaced apart from one another so as to provide a high degree of flexibility between articulating module 506 and anchor portion 504, and cap connectors 532 are spaced apart from one another so as to provide a high degree of flexibility between articulating module 506 and cap portion 502. For example, anchor connectors 534 may be placed on one of every five or six peaks 536 of distal column 537 of articulating module 506, and cap connectors 536 may be placed on one of every five or six valleys 538 of proximal column 539 of articulating module 506, such that anchor connectors 534 and cap connectors 536 are alternatingly positioned along body 530. In some embodiments, the struts of body 530 of articulating module 506 are shorter than the struts of protruding elements 509 of cap portion 502. In some embodiments, anchor connectors 534 and cap connectors 536 are straight connectors, as shown in FIG. 6. In other embodiments, anchor connectors 534 and cap connectors 536 are curved connectors, spiral connectors, or S-shaped connectors. In some embodiments, anchor connectors 534 and cap connectors 536 are pre-shaped. In some embodiments, anchor connectors 534 do not have the same configuration as cap connectors 536.

The linear compensation function of the device 500 operates primarily by function of the body 530. As shown in FIG. 6, each of the cap connectors 532 and the anchor connectors 534 is a straight connector and, therefore, connector space A=0 and does not provide a linear compensation function. When the force, or linear movement, is conveyed to the body 530, however, the sinusoidally disposed struts of the body 530 operate in a fashion that is analogous to that described with reference to FIGS. 11A and 11B.

The total linear absorption value L can then be expressed as L=2*A+Δ and, where A=0, L=Δ. In the exemplary embodiment of the device 500, L is in the range of 1-1.5 mm.

It should be readily apparent that different numbers of connectors as well as different configurations of struts, connectors, and protruding elements and patterns related thereto may vary, and that all such possibilities are within the scope of the present invention. It should also be apparent that the view shown herein represents a structure of intraluminal device 500 prior to shaping. Intraluminal device 500 may subsequently be shaped in accordance with known techniques, such that multiple protruding elements 509 are outwardly projected, forming a substantially trumpet-like configuration. A trumpet shape formed by shaping of intraluminal device 500 may have a radius of curvature in a range of 0.5-10 mm, and an angle of bending in a range of 90-180 degrees. Further, the shape and spacing of the body 530, cap connectors 532 and the anchor connectors 534 can be adjusted to modify the total linear absorption value L.

The designs of the intraluminal devices described above with respect to the various embodiments, including an articulating module disposed between cap and anchor portions, allows for various angles of articulation between each of the three portions of the device as well as energy absorption or dampening as between the cap portion and the anchor portion. This allows for use of intraluminal devices such as the ones disclosed herein at bifurcations of varying angles with increased wall apposition. Variability in flexibilities may be accomplished by shortening and/or lengthening of articulating modules, and by increasing or decreasing of strut widths within articulating modules.

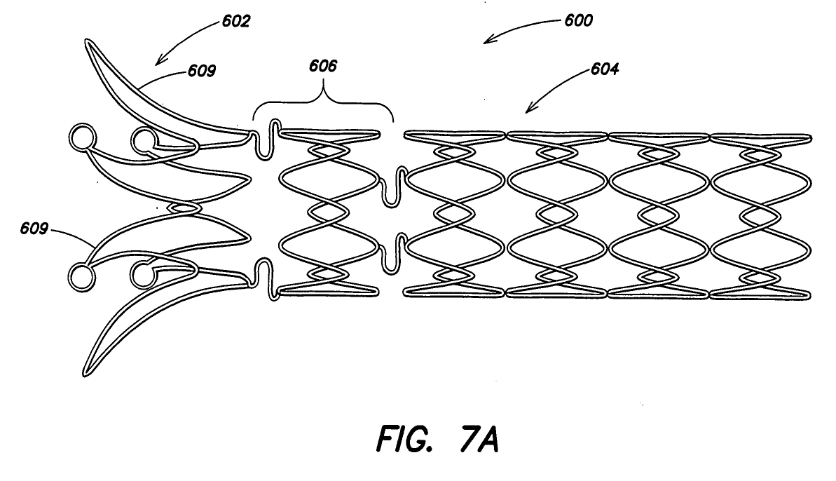

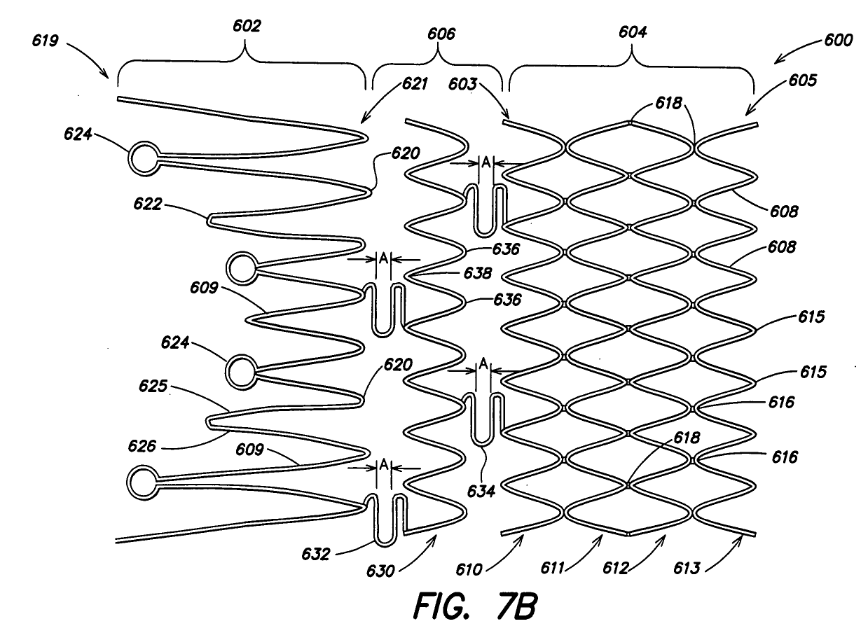

Reference is now made to FIGS. 7A and 7B, which are a perspective view and a flattened view illustration, respectively, of an intraluminal device 600 in accordance with another embodiment of the present invention. Intraluminal device 600 includes an anchor portion 604, a cap portion 602 and an articulating module 606 connecting anchor portion 604 to cap portion 602. The anchor portion 604 has an anchor portion proximal end 603 and an anchor portion distal end 605, wherein anchor portion proximal end 603 is at least partially connected to other portions of intraluminal device 600 as described hereinbelow. Anchor portion 604 is comprised of struts or supporting elements 608, which are interconnected to provide support to an inner portion of the side branch vessel 206. In some embodiments, supporting elements 608 form a uniform or repeating cell pattern, such as repeating diamond shapes, hexagonal shapes, or any other pattern. In alternative embodiments, supporting elements 608 form non-uniform patterns, having variations in pattern dimensions and/or strut characteristics. In one embodiment, supporting elements 608 are configured in a series of interconnected columns, for example, columns 610-613 shown in FIG. 7B.

It should be readily apparent that the number of columns 610-613 may vary, and that the number of columns shown and described herein with respect to the present embodiment is for illustrative purposes only. Each column 610-613 has a sinusoidal pattern having peaks 615 and valleys 616, wherein peaks 615 are defined as elements protruding in a direction facing anchor portion distal end 605 and valleys 616 are defined as elements protruding in a direction facing anchor portion proximal end 603. Adjacent columns are 180 degrees out of phase in their sinusoidal patterns, such that a peak 615 of one column, for example column 610, is in line with a valley 616 of an adjacent column, for example column 611. This configuration can be repeatedly applied to additional columns, such that any desired number of columns may be included. Columns 610-613 are connected to one another at contact areas 618 between peaks 615 of one column and valleys 616 of an adjacent column. In alternative embodiments, adjacent columns are in phase with one another, or out of phase by other degrees. A length of anchor portion 604 may be in a range of 4-20 mm when in an unexpanded state, and a diameter in a range of 2-6 mm in a fully expanded state.

Cap portion 602 includes multiple protruding elements 609 extending in a proximal direction, wherein multiple protruding elements 609 are configured to extend into or in a direction of ostial region 208. The number of multiple protruding elements 609 is chosen based on the particular anatomy in which intraluminal device 600 is to be placed. Furthermore, the lengths of each of multiple protruding elements 609 may vary, thus providing an asymmetrical cap portion 602. For example, the lengths of multiple protruding elements 609 may vary so as to form an angled edge of intraluminal device 600. For example, longest multiple protruding elements 609 may be in a range of 4-10 mm in length, while shortest multiple protruding elements may be in a range of 1-5 mm in length. These configurations allow for better protection of ostial region 208 at bifurcations of various angles. In some embodiments, the lengths of multiple protruding elements are designed for optimal coverage of a bifurcation in a range of 30-60 degrees, wherein longest multiple protruding elements are in a range of 6-10 mm in length, and shortest multiple protruding elements are in a range of 3-5 mm in length. In other embodiments, the lengths of multiple protruding elements are designed for optimal coverage of a bifurcation in a range of 10-45 degrees, wherein longest multiple protruding elements are in a range of 6-12 mm in length and shortest multiple protruding elements are in a range of 1-5 mm in length. In other embodiments, the lengths of multiple protruding elements are designed for optimal coverage of a bifurcation in a range of 60-90 degrees, wherein longest multiple protruding elements are in a range of 4-10 mm in length and shortest multiple protruding elements are also in a range of 4-10 mm in length. It should also be apparent that the view shown herein represents a structure of intraluminal device 600 prior to shaping. Intraluminal device 600 may subsequently be shaped in accordance with known techniques, such that multiple protruding elements 609 are outwardly projected, forming a substantially trumpet-like configuration. A trumpet shape formed by shaping of intraluminal device 600 may have a radius of curvature in a range of 0.5-10 mm, and an angle of bending in a range of 90-180 degrees.

An articulating module 606 is provided between anchor portion 604 and cap portion 602, and includes a body 630, cap connectors 632 and anchor connectors 634. A purpose of articulating module 606 is to provide a small radius of curvature between anchor portion 604 and cap portion 602, so that intraluminal device 600 can bend at many different angles without significant additional rotation. A further purpose of articulating module 606 is to provide a spring-like mechanism for correction of axial positioning of cap portion 602 within a vessel. Thus, a portion of a force exerted on, for example, the cap portion 602 that causes the cap portion 602 to move in a direction along the longitudinal axis of the device 600 toward the anchor portion 604, is absorbed by the articulating module 606 and the linear motion or movement is not passed along to the anchor portion 604. This “energy absorbing” or linear position compensation operation facilitates placement of the device 600, as will be described in more detail below. The energy is absorbed by operation of the body 630, cap connectors 632 and the anchor connectors 634 either individually or in combination with one another.

Body 630 may have a similar geometric pattern or configuration as anchor portion 604, or may have a different pattern or configuration. A length of body 630 is minimized so as to ensure maximum flexing capabilities. For example, a length of body 630 may be in a range of 0.5-4 mm. In one embodiment, body 630 includes a row of interconnecting struts having a sinusoidal pattern having peaks 636 and valleys 638, wherein peaks 636 are defined as elements protruding in a direction facing anchor portion 604 and valleys 638 are defined as elements protruding in a direction facing cap portion 602, as shown in FIG. 7B.

In the embodiment shown in FIG. 7B, anchor connectors 634 are disposed between peaks 636 of articulating module 606 and valleys 616 of anchor portion 604. Furthermore, cap connectors 632 are disposed between valleys 638 of articulating module 606 and peaks 620 of cap portion 602. In exemplary embodiments, anchor connectors 634 are spaced apart from one another so as to provide a high degree of flexibility between articulating module 606 and anchor portion 604, and cap connectors 632 are spaced apart from one another so as to provide a high degree of flexibility between articulating module 606 and cap portion 602. For example, anchor connectors 634 may be placed on one of every five or six peaks 636 of articulating module 606, and cap connectors 636 may be placed on one of every five or six valleys 638 of articulating module 606, such that anchor connectors 634 and cap connectors 636 are alternatingly positioned along body 630. In some embodiments, the struts of body 630 of articulating module 606 are shorter than the struts of protruding elements 609 of cap portion 602. In some embodiments, anchor connectors 634 and cap connectors 636 are straight connectors. In other embodiments, anchor connectors 634 and cap connectors 636 are curved connectors, spiral connectors, or S-shaped connectors, as shown in FIG. 7B. In some embodiments, anchor connectors 634 and cap connectors 636 are pre-shaped. In some embodiments, anchor connectors 634 do not have the same configuration as cap connectors 636.

The linear compensation function of the device 600 operates by function of the cap connectors 632, the anchor connectors 634 and the body 630. As shown in FIG. 7B, each of the cap connectors 632 and the anchor connectors 634 includes a connector space A. When a force is exerted on the cap portion 602, the space A closes, i.e., is compressed, and the portions of the cap connectors 632 or anchor connectors 634 are urged toward one another to accommodate movement of the cap portion 602. A portion of the total linear absorption provided by the cap connectors 634 and the anchor connectors is, therefore, 2*A.

Further, linear compensation is provided by the body 630. When the force, or linear movement, is conveyed to the body 630, the sinusoidally disposed struts of the body 630 will operate in a fashion that is analogous to that described with reference to FIGS. 11A and 11B.

A total linear absorption value L can then be expressed as L=2*A+Δ. In an exemplary embodiment L is in the range of 1-2 mm.

Of course, one of ordinary skill in the art will understand that there is an upper limit to how much linear movement or displacement of the cap portion 602 can be absorbed by the articulating module 606.

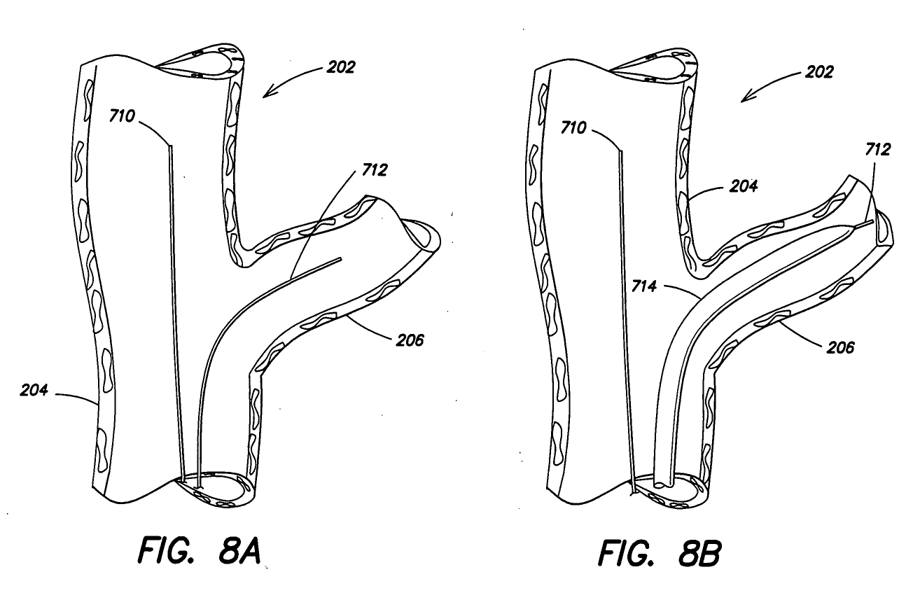

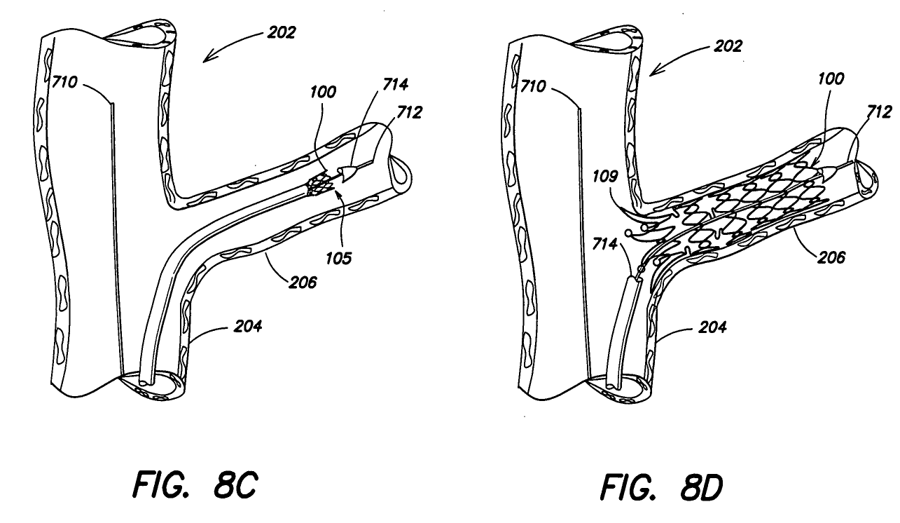

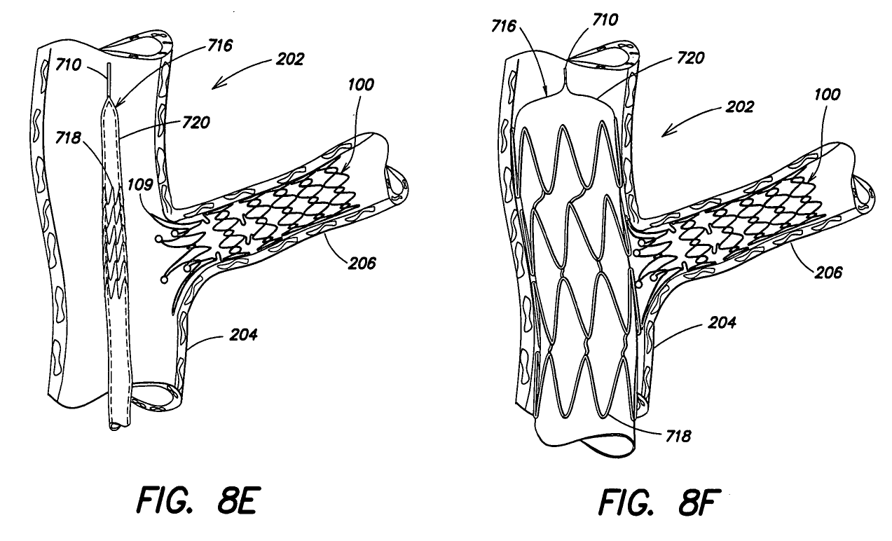

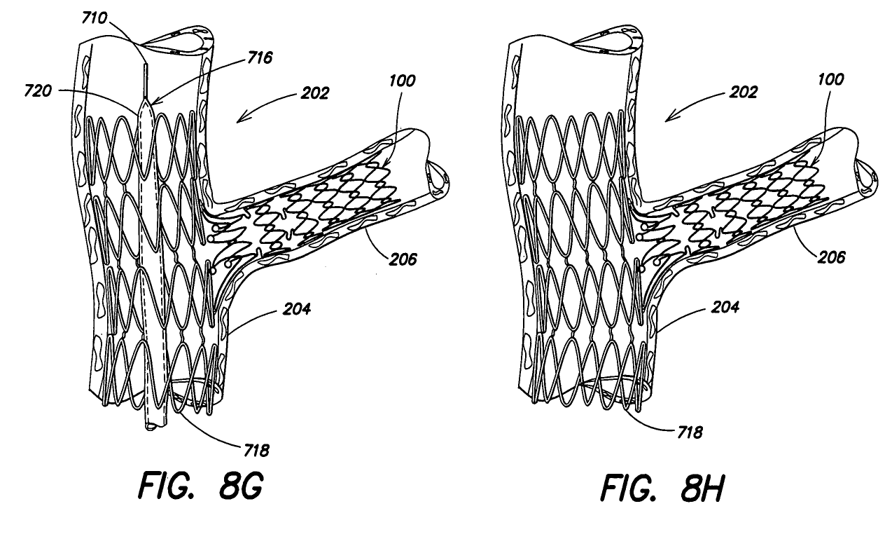

Reference is now made to FIGS. 8A-8H, which are schematic illustrations showing the steps of a method of deploying an intraluminal device 100 in accordance with exemplary embodiments of the present invention. Reference is made to FIG. 8A, which is an illustration of bifurcated vessel 202 including main vessel 204 and side branch vessel 206 extending from main vessel 204. A main branch guidewire 710 and a side branch guidewire 712 are introduced into main vessel 204 and side branch vessel 206 respectively. A delivery system 714 for delivery and deployment of intraluminal device 100 is introduced over side branch guidewire 712 and into side branch vessel 206, as shown in FIG. 8B. Delivery system 714 may be any suitable delivery system such as a sheath deployment catheter, a balloon deployment catheter or any other system suitable for delivery of intraluminal device 100.

Reference is now made to FIG. 8C, which is an illustration of delivery system 714 during deployment of intraluminal device 100. As shown in FIG. 8C, a distal portion 105 of intraluminal device 100 is released from delivery device 714 first. However, it should be readily apparent that a proximal portion of intraluminal device 100 may be released first, or the entire device may be released substantially simultaneously, as well. Intraluminal device 100 is shown after deployment in FIG. 8D. Multiple protruding elements 109 protrude into the ostial region, providing coverage in difficult to reach locations. Following deployment, delivery system 714 is removed from side branch vessel 206. In one embodiment, multiple protruding elements 109 are pre-shaped so as to retain the shape of the ostial region upon deployment. I

In one embodiment, a stent is introduced into the main vessel, and is configured to flatten at least some of the multiple protruding elements 109 against a wall of the vessel. Thus, a main vessel stent delivery device 716 is introduced into main vessel 204, as shown in FIG. 8E, for deployment of a main vessel stent 718 therein. A balloon 720 is inflated, thereby inflating stent 718, as shown in FIG. 8F. In alternative embodiments, stent 718 is a self-expandable stent and is deployed by methods other than balloon expansion, such as a removable sheath, for example. In one embodiment, stent 718 is a stent with a side hole for unblocked access to side branch vessel 206. Deployment of stent 718 causes at least some of protruding elements 109 to be compressed against the wall of the vessel. After deployment of stent 718, balloon 720 is deflated, as shown in FIG. 8G, and stent delivery device 716 is removed from the vessel, leaving intraluminal device 100 and stent 718 in place, as shown in FIG. 8H. Similar to that described above, the articulating module 106 absorbs the force of the balloon 720 and the stent 718 and prevents a portion of that force from being transmitted to the anchor portion 104. Advantageously, good opposition around the ostial region is achieved and the location of the device 100 in the side branch remains substantially undisturbed.

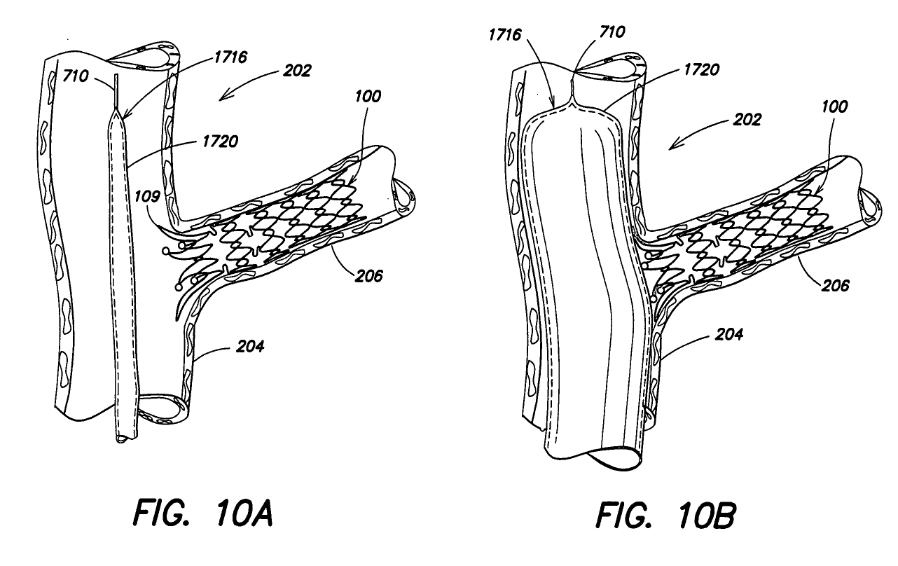

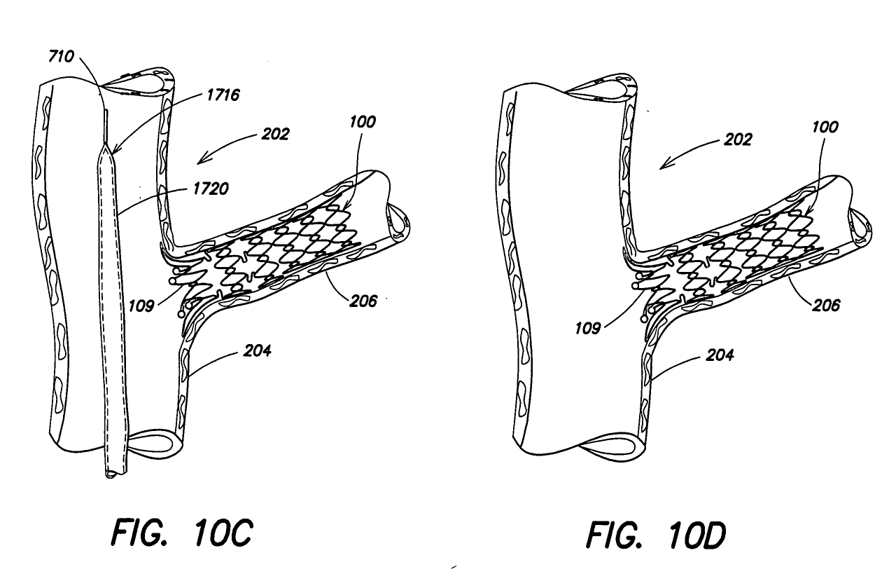

In another embodiment, intraluminal device 100 is a stand-alone device, and no further stenting is performed, however, a balloon catheter is used to flatten at least some of the multiple protruding elements 109. In this embodiment, the method steps corresponding to FIGS. 8A-8D are first performed. Subsequent to the step corresponding to FIG. 8D, a balloon catheter 1716 is inserted into the main vessel following along the guidewire 710, as shown in FIG. 1A. Next, a balloon 1720 is inflated, as shown in FIG. 10B. The expansion of the balloon 1720 causes at least some of the protruding elements 109 to be compressed against the wall of the vessel. Next, the balloon 1720 is deflated, as shown in FIG. 10C, and the balloon catheter 1716 is removed from the vessel, leaving intraluminal device 100 in place, as shown in FIG. 10D. The balloon 1720 operates to flatten at least some of the multiple protruding elements 109 against the vessel wall around the ostial region. The articulating module 106 absorbs the force of the balloon 1720 and prevents a portion of that force from being transmitted to the anchor portion 104. Advantageously, good opposition around the ostial region is achieved and the location of the device 100 in the side branch remains substantially undisturbed.

The foregoing example of the method corresponding to FIGS. 8A-8D and 10A-10D was described with respect to the device 100 for ease of explanation only and not meant to be limited to only that embodiment of the device. One of ordinary skill in the art will understand that the method is applicable to the other device embodiments as well.

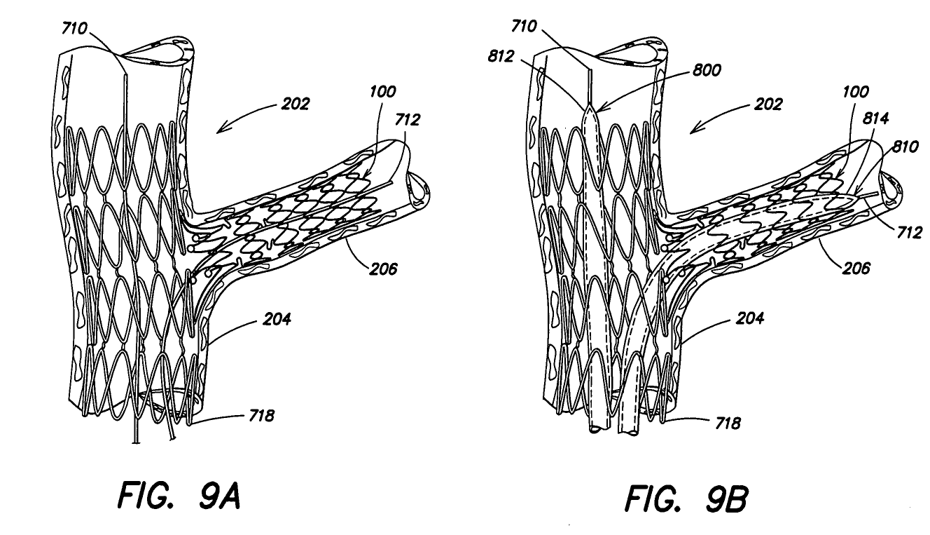

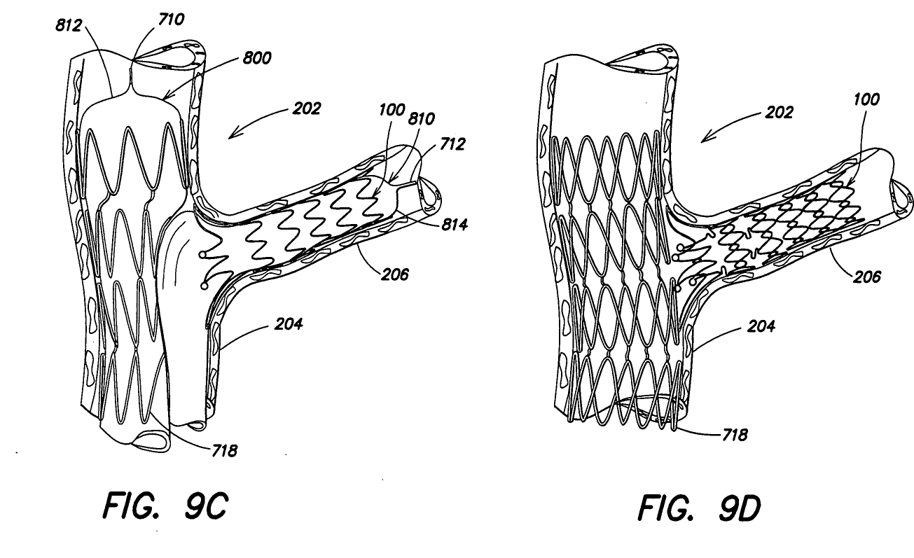

Following deployment of intraluminal device 100 and stent 718 as described above with reference to FIGS. 8A-8H, in some instances it may be desirable to increase side branch vessel access, particularly in a case where stent 718 does not have a side hole. Reference is now made to FIGS. 9A-9D, which are schematic illustrations of the steps of a method of increasing side branch vessel access. First, a main vessel guidewire 710 and a side branch vessel guidewire 712 are introduced into main vessel 204 and side branch vessel 206, respectively, as shown in FIG. 9A. Next, a main vessel balloon catheter 800 and a branch vessel balloon catheter 810 are introduced over main vessel guidewire 710 and side branch vessel guidewire 712, as shown in FIG. 9B. A main vessel balloon 812 is positioned on main vessel balloon catheter 800 and a branch vessel balloon 814 is positioned on branch vessel balloon catheter 810. As shown in FIG. 9C, main vessel balloon 812 and branch vessel balloon 814 are both inflated, preferably simultaneously, in a technique known as “kissing balloons”. This inflation causes struts surrounding ostial region 208 to deform in a way such that an opening at the ostium is increased. Main vessel balloon 812 and branch vessel balloon 814 are deflated, and main vessel balloon catheter 800 and branch vessel balloon catheter 810 are removed from the vessels, leaving only intraluminal device 100 and stent 718 in place, as shown in FIG. 9D. In this embodiment as well, the articulating module 106 prevents a portion of the force applied by any of the balloons and/or stent from being transmitted to the anchor portion 104. Advantageously, good opposition around the ostium is achieved and the location of the device 100 in the side branch remains substantially undisturbed.

Although some embodiments of the invention described above may refer to an intraluminal device configured for capping a bifurcated coronary vessel and for dispensing medication, it will be appreciated by those skilled in the art that the intraluminal device according to other embodiments of the invention may be configured for capping any other bifurcated lumen, artery or vessel, e.g., in the vascular, biliary, genitourinary, gastrointestinal and respiratory systems, which may have narrowed, weakened, distorted, or otherwise deformed, and/or for dispensing any other substance across at least part of the lumen, artery or vessel, e.g., the carotid artery or trachea bifurcations.

The medicinal coating can include, e.g., and not meant to be limiting, any one or more of the following: paclitaxol, rapamyacin, and heparin.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

What is claimed is:

1. A device for implantation in a vessel, the device comprising:
an anchor portion having a proximal end, a distal end, and an anchor body connecting said proximal and distal ends, said anchor body comprising a series of struts configured to exert a radial force;
a cap portion positioned proximal to said anchor portion;
a plurality of protruding elements disposed at a proximal end of the cap portion; and
an articulating module positioned proximal to the anchor portion and distal to the cap portion, the articulating module having a module body;
at least one compressible cap connector connecting said module body to said cap portion; and
and at least one compressible anchor connector connecting said module body to said proximal end of the anchor portion.

2. The device of claim 1, wherein:
the at least one compressible cap connector comprises an S-shaped connector having a compressible space A disposed between portions of the S-shaped connector.

3. The device of claim 2, wherein:
the at least one compressible anchor connector comprises an S-shaped connector having a compressible space A disposed between portions of the S-shaped connector.

4. The device of claim 3, wherein said body of said articulating module comprises multiple columns of struts connected by articulating body connectors.

5. A device for implantation in a vessel, the device comprising:
an anchor portion having a proximal end, a distal end, and an anchor body connecting said proximal and distal ends, said anchor body comprising a series of struts configured to exert a radial force;
a cap portion positioned proximal to said anchor portion; and
articulating means, disposed between the anchor portion and the cap portion, for absorbing a portion of linear movement of the cap portion toward the anchor portion such that the absorbed portion of the linear movement of the cap portion is not transmitted to the anchor portion.

6. The device of claim 5, wherein the articulating means comprise:
a module body;
cap connecting means for connecting said module body to said cap portion; and
anchor connecting means for connecting said module body to said anchor portion.

7. The device of claim 6, wherein:
one or more of the module body, cap connecting means and anchor connecting means absorbs the portion of the linear movement of the cap portion.

8. A method of implanting a device in a vessel, wherein the device comprises:
an anchor portion having a proximal end, a distal end, and an anchor body connecting said proximal and distal ends, said anchor body comprising a series of struts configured to exert a radial force;
a cap portion positioned proximal to said anchor portion;
a plurality of protruding elements disposed at a proximal end of the cap portion; and
an articulating module positioned proximal to the anchor portion and distal to the cap portion, the articulating module having a module body;
at least one compressible cap connector connecting said module body to said cap portion; and,
and at least one compressible anchor connector connecting said module body to said proximal end of the anchor portion,
the method comprising:
positioning the anchor portion of the device in a side branch vessel such that the struts of the anchor body exert a radial force on the side branch vessel and the cap portion is positioned at a bifurcation location between the side branch vessel and a main branch vessel;
exerting a force upon the cap portion to urge the cap portion a linear distance toward the anchor portion; and
absorbing a portion of the linear distance with the articulating module such that the anchor portion substantially remains in position within the side branch vessel.

9. A device for implantation in a vessel, the device comprising:
an anchor portion having a proximal end, a distal end, and an anchor body connecting said proximal and distal ends, said anchor body comprising a series of struts configured to provide a radial force to a wall of the vessel;
a cap portion positioned proximal to said anchor portion, said cap portion comprised of multiple protruding elements for extension into an ostial region of said vessel; and
an articulating module having a body, at least one cap connector for connecting said body to said cap portion, and at least one anchor connector for connecting said body to said anchor portion,
wherein said at least one cap connector is configured for flexing in a first direction and said at least one anchor connector is configured for flexing in a second direction.

10. The device of claim 9, wherein said first direction is substantially orthogonal to said second direction.

11. The device of claim 9, wherein at least one of said multiple protruding elements is longer than at least another one of said multiple protruding elements.

12. The device of claim 9, wherein said multiple protruding elements are configured from upper segments and lower segments forming peaks and valleys, and wherein said body of said articulating module has a repeating pattern including peaks and valleys, wherein said at least one cap connector connects a valley of said body to a peak of said cap portion, and wherein said at least one anchor connector connects a peak of said body to a valley of said anchor portion.

13. The device of claim 9, wherein said multiple protruding elements form a trumpet shape upon deployment of said device.

14. The device of claim 9, wherein said device is comprised of at least one of: a super-elastic material; a shape-memorizing material; an elastic material.

15. The device of claim 9, wherein said at least one cap connector comprises two cap connectors separated from one another by approximately 180 degrees around said body of said articulating module.

16. The device of claim 9, wherein said at least one anchor connector comprises two anchor connectors separated from one another by approximately 180 degrees around said body of said articulating module.

17. The device of claim 9, wherein at least part of said device is coated with at least one layer of a desired medication.

18. The device of claim 9, wherein at least one of said multiple protruding elements is longer than said struts of said anchor portion.

19. The device of claim 9, wherein said anchor portion is comprised of multiple anchor sections.

20. A device for implantation in a vessel, the device comprising:
an anchor portion having a proximal end, a distal end, and an anchor body connecting said proximal and distal ends, said anchor body comprising a series of struts configured to provide a radial force to a wall of the vessel;
a cap portion positioned proximal to said anchor portion, said cap portion comprised of multiple protruding elements for extension into an ostial region of said vessel; and
articulating means, coupled between the anchor portion and the cap portion, for flexing in a first direction and in a second direction.

21. The device of claim 20, wherein said first direction is substantially orthogonal to said second direction.

22. The device of claim 20, wherein said multiple protruding elements are configured from upper segments and lower segments forming peaks and valleys, and wherein said body of said articulating module has a repeating pattern including peaks and valleys, wherein said at least one cap connector connects a valley of said body to a peak of said cap portion, and wherein said at least one anchor connector connects a peak of said body to a valley of said anchor portion.

23. he device of claim 20, wherein said multiple protruding elements form a trumpet shape upon deployment of said device.

24. The device of claim 20, wherein said device is comprised of at least one of: a super-elastic material; a shape-memorizing material and an elastic material.

25. The device of claim 20, wherein said articulating means comprise two cap connectors coupled to the cap portion and separated from one another by approximately 180 degrees around said device.

26. The device of claim 20, wherein said articulating means comprise two anchor connectors coupled to the anchor portion and separated from one another by approximately 180 degrees around said device.

27. The device of claim 20, wherein at least part of said device is coated with at least one layer of a desired medication.

28. The device of claim 20, wherein at least one of said multiple protruding elements is longer than said struts of said anchor portion.

29. The device of claim 20, wherein said anchor portion is comprised of multiple anchor sections.

30. A method for providing coverage to an ostial region of a bifurcated vessel, the method comprising:
providing a device including an anchor portion, a cap portion positioned proximal to said anchor portion, said cap portion comprised of multiple protruding elements, and an articulating module including a body, at least one cap connector for connecting said body to said cap portion, and at least one anchor connector for connecting said body to said anchor portion;
introducing said device into a side branch of said bifurcated vessel;
at least partially releasing said anchor portion for anchoring said device in said side branch; and
releasing said cap portion such that said multiple protruding elements extend into the ostial region, wherein said extension includes providing an angular shift in accordance with an anatomy of the ostial region.

31. The method of claim 30, further comprising:
introducing a stent into a main branch of the bifurcated vessel; and
compressing at least some of said multiple protruding elements.

32. The method of claim 31, further comprising:
introducing a main vessel balloon into a main vessel of the bifurcation and a side branch vessel balloon into a side branch vessel of the bifurcation;
inflating said main vessel balloon and said side branch vessel balloon;
deflating said main vessel balloon and said side branch vessel balloon; and
removing said main vessel balloon and said side branch vessel balloon from the main vessel and side branch vessel, respectively.
